(19) United States
(12) Patent Application Publication
(10) Pub. No.: US 2009/0030453 A1
(43) Pub. Date: Jan. 29, 2009

(54) Surgical Device

(75) Inventor: Jassim Daood, Potts Point (AU)

Correspondence Address: FOLEY HOAG, LLP;PATENT GROUP, WORLD TRADE CENTER WEST, 155 SEAPORT BLVD, BOSTON, MA, 02110, US

(73) Assignee: AESTHETIC DEVICES PTY LIMITED, Crows Nest, New South Wales (AU)

(21) Appl. No.: 12/281,095

(22) PCT Filed: Feb. 27, 2007
(86) PCT No.: PCT/AU2007/000229

§ 371 Date: Sep. 22, 2008

(30) Foreign Application Priority Data
Feb. 28, 2006 (AU) 2006901003

(51) Int. Cl. A61B 17/06

(52) U.S. Cl. 606/223

(57) ABSTRACT

A surgical needle (10) that has two skin penetrating tips 14a and (14b) and an elongate single eye (15) that extends along a majority of the length of the needle. The needle is used to introduce and advance a surgical device (30) subcutaneously.

### CROSS-REFERENCE TO RELATED APPLICATIONS

The present application claims priority from Australian Provisional Patent Application No 2006901003 filed on 28 Feb. 2006, the content of which is incorporated herein by reference.

### FIELD OF THE INVENTION

The present invention relates to a needle and an apparatus for use in surgery. The needle and device may have particular application in plastic or cosmetic surgery.

### BACKGROUND ART

The human skin is a vital organ that performs a number of functions essential for the health and wellbeing of individuals. As well as acting as a barrier protecting the individual from the environment, skin helps regulate body temperature, aids in fluid and electrolyte balance, and provides receptors for sensations such as touch, pain and pressure.

In this regard, skin can be considered to consist of three layers: an outer layer (epidermis) which contains the skin cells, pigment and proteins; the middle layer (dermis) which contains the blood vessels, nerves, hair follicles and oil glands; and the inner layer (subcutaneous) layer which contains sweat glands, some hair follicles, blood vessels and fat. Each layer contains connective tissue having collagen fibres to give support to the skin and elastin fibres to provide flexibility and strength to the skin.

As an individual ages, the epidermis thins and the connective tissue changes thereby reducing the skin's strength and elasticity. The skin begins to lose its elastin fibers and the fibers become tangled in disorganised masses as the blood vessels in the skin shrivel. Thinning and degeneration of elastin over time causes sagging of the skin, as does the loss of collagen in the skin, which loses collagen at a rate of about one percent each year.

Such sagging and drooping of the skin combined with constant exposure to sunlight and the effects of gravity can be most noticeable in the faces of individuals. This is typically in the form of deep crease or wrinkles forming around the nose, eyes and mouth; the jawline becoming slack and jowly; and folds and fat deposits appearing around the neck of the individual.

A variety of methods and treatments have been proposed to treat the signs of ageing, however no treatment or method can stop the ageing process. Proposed methods and treatments include applying toning creams and ointments to the region of the affected skin, injecting collagen or body fat harvested from other areas of the individual's body into the affected region via a small hypodermic needle, peeling or resurfacing by chemical or laser treatment the top layer of the skin to encourage new skin regrowth; and injecting a toxin such as Botulinum toxin (Botox®) in to the affected area to paralyse the muscles to prevent them from wrinkling the skin.

For more permanent treatments of ageing skin, plastic or cosmetic surgery is possible which are generally performed under general anaesthetic or in some instances local anaesthetic combined with a sedative. Typically such surgeries require separating the skin from the fat and muscle below and trimming and suctioning away some of the fat, tightening the underlying muscle and membrane, pulling the skin back, and removing the excess. Stitches are typically used to secure the layers of tissue and close the associated incisions. In any regard, a facelift or Rhytidectomy is a relatively complicated procedure requiring much surgical expertise and significant recuperation following surgery.

There is therefore a need to provide a device which can simplify the surgical process.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect, the present invention is a surgical needle comprising:

an elongate body having;
a first end and a second end, each end comprising a skin penetrating tip; said elongate body defining an eye extending at least a majority of the length of the elongate body between said first end and said second end.

The needle of the present invention has the advantage that it enables a user to advance a medical device subcutaneously. As will be described in more detail below, the device that is advanced may comprise a range of structures including sutures of various types and implantable devices.

Typically, the length of the eye of the needle is greater than approximately 50% of the length of the elongate body between the two ends. In a preferred embodiment, the length of the eye of the needle is greater than or equal to 60%, more preferably greater than or equal to 70%. In a further preferred embodiment, the length of the eye is greater than or equal to 80% of the length of the elongate body. Still further, the length of the eye may be greater than or equal to 90% of the length. In yet another embodiment, the length of the eye of the needle may be greater than or equal to 95% of the length of the elongate body. In one preferred embodiment, the length of the eye may be greater than or equal to 97% of the length of the elongate body.

The widest diameter of the eye of the elongate body may be between 50% and 70% of the total width of the widest part of the elongate body. In a preferred embodiment, the width of the eye, at its widest part, is between 20% and 39% of the total width of the widest part of the elongate body. The widest part of the needle is typically mid-way between the two ends.

The elongate body may further include a series of markings thereon. The markings typically extend from around the middle of the elongate body and towards one or both ends. The marking typically denote the distance from the centre point of the elongate body. The markings may be in 1 mm increments. This is particularly useful as it enables a surgeon to position a device at a certain depth and location within a patient. Typically, therefore, the needle may be inserted into the skin at substantially right angles to the skin to give an accurate measurement of depth beneath the skin.

The needle is preferably made from a suitable biocompatible material including metals and metal alloys including a combination thereof. In one embodiment, the needle is made from stainless steel including surgical grades 316 and 420. Further, the needle may be made from a suitable polymeric material. In a further embodiment, the needle may be made from a combination of materials. One example is a combination of a suitable metal and a polymeric material.

Preferably, the needle comprises a substantially linear elongate body. Alternatively, the elongate body may be non linear. In one embodiment the elongate body may comprise a degree of curvature.

According to a second aspect, the present invention is a method of subcutaneously positioning a device within an individual, comprising;

(a) providing a surgical needle comprising an elongate body and a first end and a second end, each end comprising a skin penetrating tip; said elongate body defining an eye extending at least a majority of the length of the elongate body between said first end and said second end;

(b) positioning the device at least partially within the eye of the needle;

(c) subcutaneously introducing the device by passing one of the first end or second end of the needle into and under the skin of the individual;

(d) moving said needle relative to the skin such that said one end exits the skin at a position remote from the position at which the said one end entered and wherein the other of said first and second ends and at least a portion of the device is retained beneath the surface of the skin;

(e) passing said other end of the needle subcutaneously and causing it to exit the skin at a position remote from the position at which the said one end exited the skin of the individual and wherein the said one end and at least a portion of the device is retained beneath the surface of the skin; and

(f) if required, repeating steps (d) and (e) to further subcutaneously advance the device.

The needle allows the device to be positioned subcutaneously and to remain in a subcutaneous position as it is tunnelled through surrounding tissue. At any given time during the procedure, one of the ends of the needle is beneath the skin thus retaining the device beneath the skin. It may be understood that the tunnelling of the device may be achieved as a result of the length of the eye of the needle. The device may slide along the length of the eye of the needle as the needle moves in, beneath and out of the skin such that the device remains beneath the skin at any given point in the procedure.

In a further embodiment, the needle may be maneuvered to change the direction of the end that is positioned subcutaneously. For example, as one end exits the skin, the trailing subcutaneous end is moved from a relatively rearward facing direction to a relatively forward facing direction. Pressure may then be applied to the needle to cause what was the trailing end to pass subcutaneously and to exit the skin at a position remote from the entry point.

In another aspect, the invention provides a needle assembly comprising:

a surgical needle comprising an elongate body having a first end and a second end, each end comprising a skin penetrating tip; said elongate body defining an eye extending at least a majority of the length of the elongate body between said first end and said second end; and

an implantable device receivable in the eye of the needle.

The needle of this aspect may have any one or more of the features of the needle described above.

The surgical needle of the present invention may be used to deliver a number of different devices. In one embodiment, the device may be a tissue tensioning device.

The tissue tensioning device may be made of suture material and comprise a suture member. In this embodiment, the device may include a tissue engaging member to engage a region of tissue adjacent to an entry point of the needle. The remainder of the device may be advanced away from the entry point, to a distal region whereupon tension may be applied to the device and thus the tissue with which the device is engaged.

The suture member may include a series of spaced apart barb members along at least a portion of its length to engage with surrounding tissue. When the suture member is tensioned, the barb members pull on the tissue to tighten said tissue.

In a further embodiment, the tissue tensioning device comprises:

an elongate member having a first end and a second end;

a hook element attachable to the first end for engaging said tissue; and

an anchor member positionable at a region of the body remote from said tissue and configured to engage at least a portion of said elongate member at a location distal the first end such that a tension force can be generated in said elongate member and applied to said tissue.

In one embodiment, the elongate body of the tissue tensioning device comprises a plurality of engaging elements provided along a part or all of its length for engaging with the anchor means. The engaging elements may be irregularly spaced along said part or all of the length of the elongate member. The engaging elements may be irregularly spaced along a part of said length and regularly spaced along another part of said length of the elongate member.

In one form, the engaging elements may be notches or ridges formed on a surface of the elongate member. The notches or ridges may be regularly spaced along said part or all of the length of the elongate member and may be arranged orthogonal to the length of the elongate member. In this regard, the surface of the elongate member may comprise a saw tooth configuration extending substantially from the first end to the second end.

In another form, the engaging elements may be shaped regions of the elongate member. The shaped regions may be in the form of conically shaped regions arranged along part, some or all of the length of the elongate member in an end-to-end manner. Each of the conically shaped regions may have a first end having a first cross-section tapering to a second end having a second cross-section which is substantially smaller in diameter than the first cross-section. In this arrangement, the external surface of the elongate member may comprise a series of stepped regions formed at substantially regular intervals along the length of the elongate member, from adjacent the first end to adjacent the second end.

The anchor member may comprise a recess for slidingly receiving the second end of the elongate member. The anchor member may also comprise a pawl which is arranged to engage with one of the plurality of notches or ridges formed on the surface of the elongate member. In one form, the pawl may be a substantially resilient latch member that permits movement of the elongate member through the anchor member in a first direction but substantially prevents movement of the elongate member through the anchor member in a second direction.

In another form, the pawl may be a substantially resilient plate arranged to engage with a first end of the shaped regions of the elongate member to substantially prevent movement of the elongate member through the anchor member in the second direction, but permit movement of the elongate member through the anchor member in the first direction. In this regard, movement of the elongate member through the anchor member in the first direction corresponds to advancement of the elongate member through the recess and thus the generation of the tension force in the elongate member, whilst movement of the elongate member in the second direction corresponds to withdrawal of the elongate member from the anchor member and a reduction in the tension force generated in the elongate member.

In one embodiment, the anchor member is fixedly secured to the body in a region remote from the tissue being tensioned. The anchor member may be secured to a bone of the body by screws and/or cement such that the anchor member is substantially fixed in position. In another embodiment, the anchor member may be non-fixedly secured to the body. The anchor member may be positioned adjacent a tunnel formed in the tissue which is remote from the tissue being tensioned. The tunnel may act against the anchor body to substantially prevent the anchor member moving in the second direction and hence substantially prevent any reduction in tension forces being applied to the tissue.

In another embodiment, the tissue being tensioned is a facial muscle. In this embodiment, the anchor member may be positioned in the region of the skull adjacent a recipient's ear.

According to another aspect, the invention is an elongate member for use in conjunction with an anchor member to tension the tissue of a patient comprising:

a first end and a second end;

a hook element attachable to the first end for engaging said tissue; and

wherein at least a portion of said elongate member engages with the anchor member at a location distal the first end such that a tension force can be generated in said elongate member and applied to said tissue.

According to a further aspect, the invention is an anchor member for use in conjunction with an elongate member to tension the tissue of a patient comprising:

a body element positionable at a region of the body remote from said tissue and configured to engage at least a portion of said elongate member at a location distal the first end such that a tension force can be generated in said elongate member and applied to said tissue.

According to another aspect, the present invention is a surgical needle, comprising:

an elongate body having
a first end and a second end, each end being configured to penetrate the skin of the patient to enable said body to be inserted at least partially into the patient's skin; and
an eye extending substantially the length of the elongate body between said first and second end.

According to a further aspect, the present invention is a method of subcutaneously positioning a device within an individual, comprising;

(a) positioning said device within an eye of the needle according to the fourth aspect of the present invention;

(b) passing one of the first end or second end of the needle through the skin of the individual such that said one end exits the skin of the individual at a position remote from the position at which the said one end entered the individual and wherein the other of said first and second ends is retained beneath the surface of the skin;

(c) passing said other end of the needle through the skin of the individual such that the said other end exits the skin of the individual at a position remote from the position at which the said one end exited the skin of the individual and wherein the said one end is retained beneath the surface of the skin; and

(d) repeating steps (b) and (c) such that the device is subcutaneously positioned in a desired manner.

### BRIEF DESCRIPTION OF THE DRAWINGS

By way of example only, preferred embodiments of the invention are now described with reference to the accompanying drawings, in which:

FIG. 1 is a surgical needle according to the present invention;

FIGS. 2A to 2C depict various views of the process for positioning a device using the needle of the present invention;

FIG. 3 is a perspective cross-sectional view of a surgical device that may be delivered using the needle of the invention;

FIG. 4 is a plan view of the embodiment of FIG. 3;

FIG. 5 is a simplified view showing the surgical placement of the device as shown in the embodiments of FIGS. 3 and 4;

FIG. 6 is an enlarged cross sectional view of the attachment arrangement for attaching and tightening the device as shown in the embodiments of FIGS. 3 and 4;

FIGS. 7A and 7B depict top and side views respectively of an alternative embodiment of the surgical device in accordance with the present invention;

FIG. 8 depicts a perspective view of the device of FIGS. 7A and 7B;

FIG. 9 depicts a top view of the hook element of the device of FIGS. 7A and 7B;

FIG. 10 depicts a top view of the locking member of the device of FIGS. 7A and 7B in use; and

FIGS. 11a to 11c depict a surgical procedure using the needle of the present invention.

### DETAILED DESCRIPTION OF AN EXEMPLARY EMBODIMENT OF THE PRESENT INVENTION

The needle of the present invention is generally depicted as 10 in the accompanying drawings. The needle comprises an elongate body 11 that has a first end 12 and a second end 13. Each end has a skin penetrating tip 14a and 14b. The elongate body defines an eye 15. An important feature of the invention is that the eye extends along a majority of the length of the elongate body 11 between the first end 12 and the second end 13.

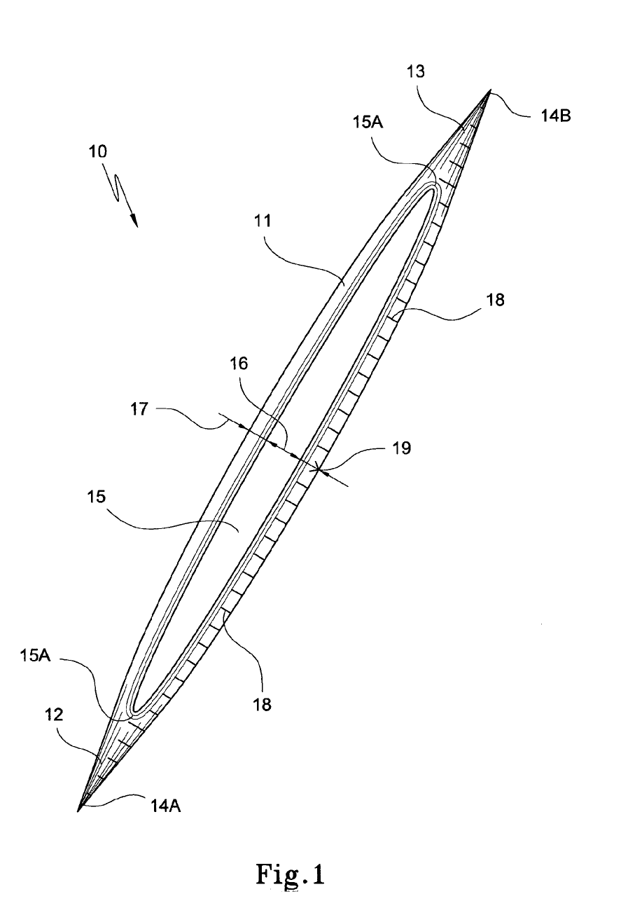

The length of the eye 15 of the needle is greater than approximately 50% of the length of the elongate body between the two ends. In the embodiment of the needle depicted in FIG. 1, the length of the eye relative to the length of the elongate body 11 is approximately 70%.

The widest diameter 16 of the eye 15 of the elongate body 11 is equal to or greater than 50% of the total width 17 of the widest part of the elongate body 11.

As depicted in FIG. 1, the elongate body 11 includes a series of markings 18 along its length. The markings extend from mid-point 19 of the elongate body 11 towards both ends 12 and 13. The markings 18 denote the distance from mid-point 19 and are typically in 1 mm increments.

The needle 10 of the present invention may be used to deliver and position a number of medical devices subcutaneously. The types of devices range from sutures to implantable devices that are used to tension tissue such as those used in corrective or plastic surgery procedures. Application of the needle is also found in wound closing and any other application that requires the delivery of a device subcutaneously wherein said device may be loaded onto the needle of the invention. The size of the needle of the present invention will vary depending upon the size and configuration of the device to be delivered.

As seen in FIG. 1, the portions of elongate body that define the eye at its ends 15a provide a smooth, curved structure, avoiding any angles or sharp edges that may snag or tear a suture or other device to be delivered.

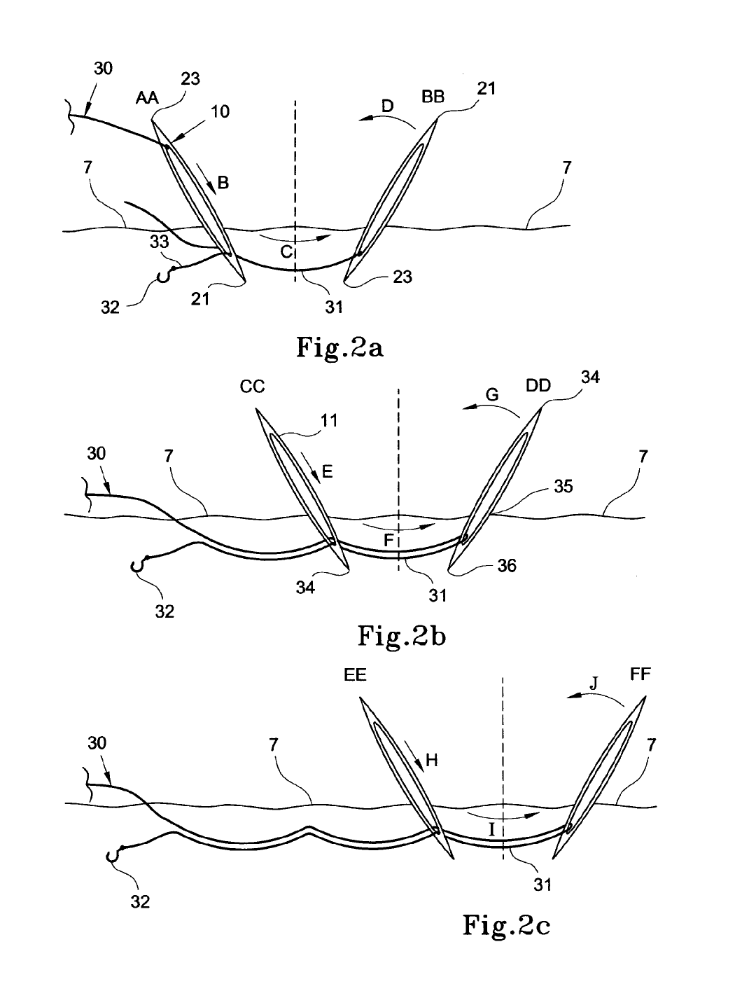

During a surgical procedure, a device is loaded onto the needle 10 of the invention. In FIGS. 2A to 2C, the device is generally depicted as 30. In this case, the device comprises a suture 31 that has a hook 32 at a proximal end 33. The hook is designed to bite into surrounding tissue and thus anchor the proximal end of the suture. In embodiments wherein the suture does not have a hook, the surgeon may anchor the proximal end of the suture by looping it around a suitably strong bodily structure.

The surgeon applies pressure to the needle 10 to puncture the skin 7. Depending upon the type of procedure being performed, the surgeon will appropriately position a leading end 21 beneath the skin 7. In a face lift (rhytidectomy) procedure, the surgeon may position the leading end 21 in or through the facial muscle of a patient such that hook 32 of device 30 is anchored in the facial muscle. The leading end 21 of the needle 10 then travels under the skin 7 along the direction of arrow C where it is pushed back through the skin 7, to be in the position BB. In position BB, the leading end 21 is exposed while trailing end 23 of the needle 10 remains subcutaneous. In the depicted embodiment, the needle 10 is essentially pivoted in the direction of arrow D to be in position CC as shown in FIG. 2B. In position CC, the needle is primed for the next stroke, that is pressure is applied to the elongate body 11 (arrow E) such that, what was the trailing end 33, becomes the leading end 34 as shown in FIG. 2B. The now leading end 34 travels a distance subcutaneously (arrow F) before exiting at point 35. The leading end 34 is exposed with the now trailing end 36 positioned subcutaneously (position DD).

The suture 31 freely moves along the eye 15 of the needle 10 during the strokes of the needle and during each cycle as described above. The suture, therefore, remains in a subcutaneous position while advancing away from the initial entry point, throughout the procedure. The progressive movement of the needle creates a tunnelling effect and the suture progresses a length of the patient's body subcutaneously.

The above steps may be repeated as many times as required. For example, in a face lift procedure a smaller number of cycles of the needle are required than in a buttock lift or a breast lift where larger areas of tissue are to be tightened. If further cycles are required, the needle is moved from position DD in the direction of arrow G into position EE where the needle is pushed through the skin along the direction of arrow H and arrow I to emerge from the skin in position FF of FIG. 2C.

When the surgeon reaches a desired length of tunnelling, tension may then be applied to the suture. For example, in a face lift procedure, the distal end of the suture may be secured to tissue around the temporal region of a patient to apply tension to the suture. With the proximal end 33 of suture 31 embedded in the facial muscle, any tension applied to the suture will lift or tighten the facial muscle to the degree required.

The suture or other device may also be looped over a structure distal to the entry point and then tunnelled back to the entry point. The two ends of the device may then be tensioned to effect a lift.

An advantage of the present needle, therefore, is that it enables the positioning of a device such as a suture beneath the skin over a range of lengths without the requirement for individual incisions to position the suture. The only marks left on the patient are the needle entry and exit point which are far less likely to leave scarring.

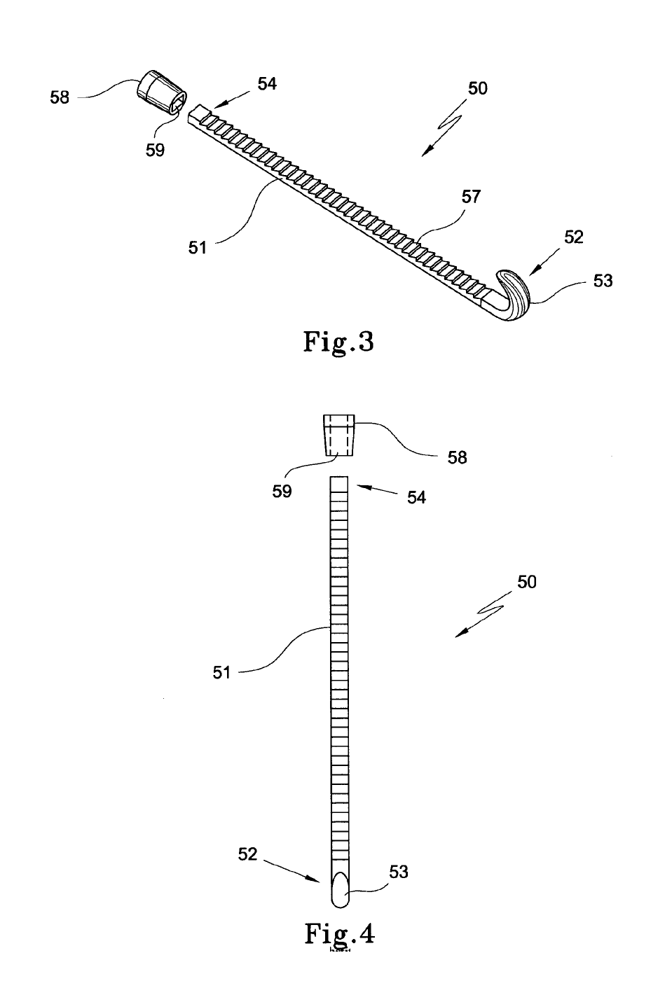

While the above description relates to the delivery of a suture type device, other implantable devices may be delivered using the needle and method described. One such device is depicted as 50 in the accompanying drawings. FIG. 3 depicts a cross sectional side view of the device 50. The device has an elongate member or body 51 having a distal end 52 and a proximal end 54. A hook 53 is provided at the distal end 52, the purpose of which will be discussed in more detail below.

In the depicted embodiment, the elongate body 51 is substantially rectangular in cross-section and comprises a plurality of regularly spaced ridges 57 formed thereon forming a saw tooth configuration extending between the distal end 52 and proximal end 54 of the body 51.

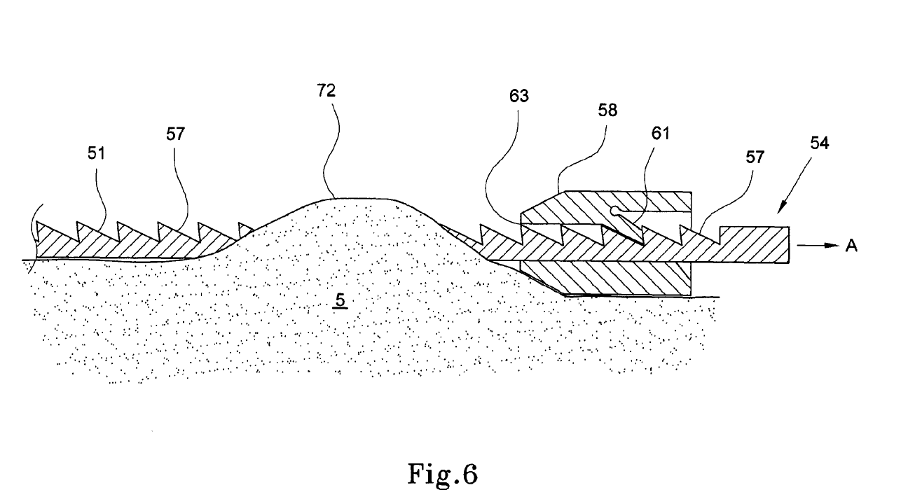

The device 50 also comprises a locking member 58 having a central recess or through hole 59 shaped to receive the body 51. As will be discussed in more detail in relation to FIG. 6, the locking member 58 receives the proximal end 54 of the body 51 and allows the body to freely pass into the and through the hole 59. Any movement in the opposite direction, i.e. in a direction to withdraw the body 10 from the locking member 58, is prevented by an internal pawl member acting against the ridges 57.

The body 51 and the locking member 58 are made from a plastics material such as a nylon or polypropylene which can be moulded to relatively fine tolerances to form the ridges 57 to the necessary degree of precision. The hook 53 may be made from a stainless steel and attached to the distal end 52 of the body 51 through crimping or the like.

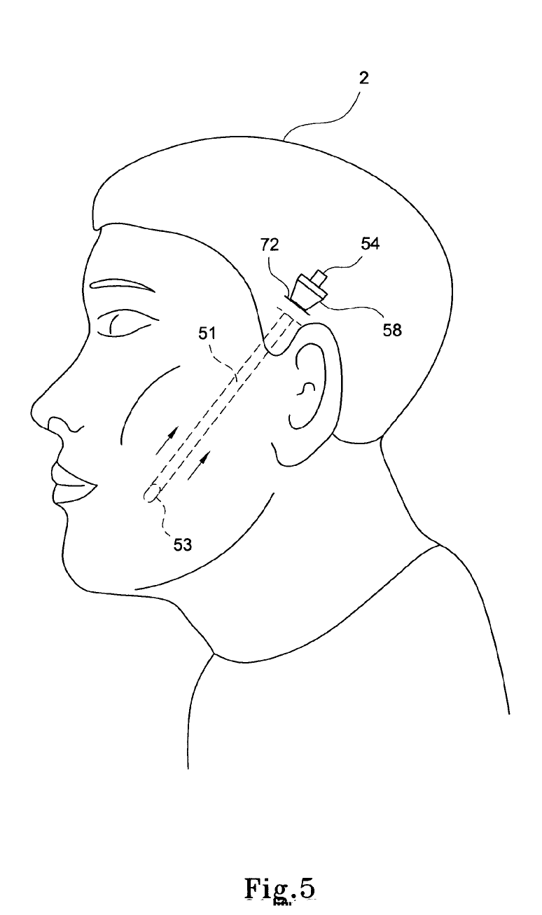

The manner in which the device 50 is used to perform a rhytidectomy is shown in FIG. 5. The device 50 is introduced into the face of the individual 2 proximal the sagging or drooping region of the individual's face. The device 50 is introduced such that the hook 53 may be either introduced first or last. Once introduced, the needle 10 is passed through the muscle proximal the sagging/drooping skin and tunnels into the muscle such that the hook 53 catches the facial muscle. Following the hook 53 anchoring in the muscle, the body 51 is tunnelled under the skin of the face up to the skull, above the ear of the individual 2 using the needle method described above.

During the rhytidectomy, the surgeon makes a small tunnel 72 through the periosteum of the skull in the region above the individual's ear, through which the proximal end 54 of the body is passed. The locking member 58 is then inserted over the proximal end 54 such that the body 50 passes through the hole 59 formed in the locking member 58. As the hook 53 is firmly embedded in the muscle at the distal end 52 of the body 51, the surgeon is able to apply a force at the proximal end 54 to cause the muscle to be pulled in an upwards direction, thereby tightening the muscle into a desired position. As force is applied against the proximal end 54 of the body 51, the locking member 58 is advanced along the body 51 such that it is against the tunnel 72 formed in the periosteum, thereby locking the device 50 into the desired position. As the locking member 58 prevents withdrawal of the body 51, tension is maintained in the body 51 such that the muscle is maintained in the desired position.

The manner in which the locking member 58 and the tunnel 52 cooperate to secure the device 50 in position is shown in detail in FIG. 6. The periosteum, namely the dense fibrous membrane covering the surface of the skull, is shown generally as 5 with the tunnel 72 formed through the periosteum such that the body 51 of the device can pass therethrough. The locking member 58 comprises a resilient latch pawl 61 which is inclined downwardly in a direction away from the entry 63 of the locking member. In this arrangement, as force is applied to the proximal end 54 of the body 10 to tighten the muscle (not shown) which is attached to the hook 53 of the distal end 52, the body 51 is advanced through the locking member 58. Advancement of the body 51 through the locking member causes the resilient latch pawl 61 to ride the various sloped surfaces of the ridges 57. Following advancement of the body 51 in the direction of arrow A to facilitate tightening of the muscle, any movement of the body 51 in a direction opposite to A is prevented by the resilient latch pawl 61 contacting the vertical face of the adjacent ridge 57.

As the locking member 58 is positioned adjacent the exit of the tunnel 72 formed in periosteum 5, both the tunnel 72 and the locking member 58 act as an anchor to prevent unwanted movement of the body 51, and hence loosening of the muscle from the desired position and tightness.

While the locking member 58 has been shown employing a latch pawl arrangement, it will be appreciated that a number of locking arrangements could also be employed to prevent unwanted movement of the body 51 away from the locking member 58.

It will also be appreciated that the device 50 as shown in FIGS. 3 to 6 can also readily facilitate further tightening procedures/lifts. In this regard, in order to perform a further facelift procedure, a small incision can be made in the region adjacent the tunnel 72 formed in the skull to access the locking member 58. The body 51 can then be further advanced through the locking member 58 along the direction of arrow A to apply further tension to the body 51 and hence the muscle attached to the hook 53, to further lift the muscle to achieve the desired result. Such a procedure can be performed with minimal inconvenience to the patient and under a local anaesthetic.

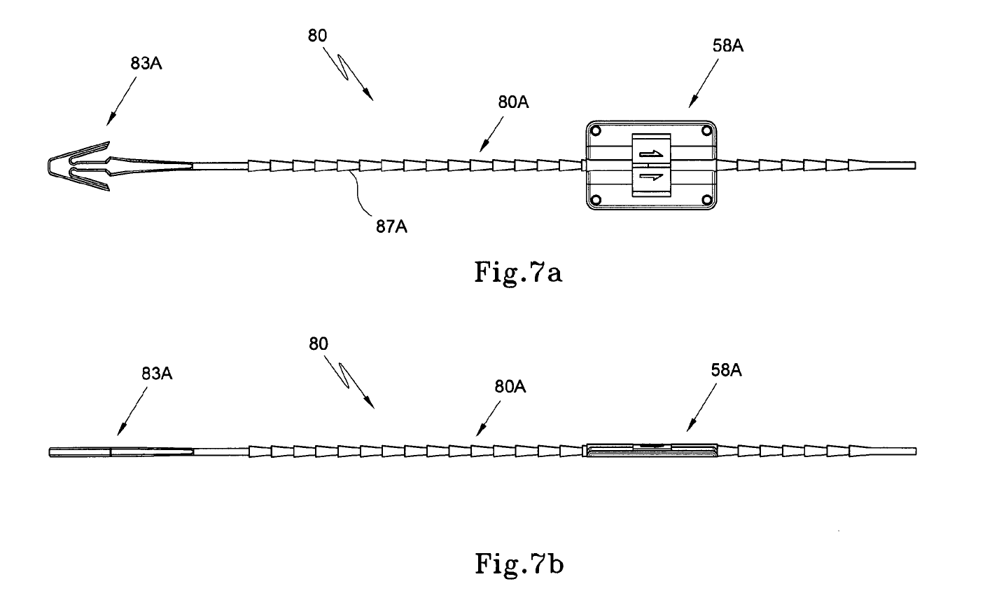

An alternative embodiment of the device of the present invention is shown in FIG. 7 to FIG. 10 and is referred to by reference numeral 80.

Referring to FIGS. 7A and 7B, the device 80 has an elongate body 80a having a hook 83a attached to one end thereof. The body 80a is substantially tubular in cross section and comprises a plurality of conical portions 87a arranged substantially along the length of the body. As is more clearly shown in FIG. 10, each conical portion 80a has a first end 81 which tapers towards a second end 82 of reduced diameter in relation to the first end 81. In this regard, as the conical portions 87a are arranged in an end-to-end relationship along substantially the length of the body 80a, the outer surface of the body 80a has a substantially stepped configuration defined by the junction where the second end 82 of one conical portion 87a meets the first end 81 of the adjacent portion 87a.

The hook 83a is shown in more detail in FIG. 9 and resembles an anchor having a pair of arms 84 extending therefrom for attaching to the muscle or other tissue requiring lifting. As shown in FIG. 7B, the arms 84 extend in only one plane, however it will be appreciated that the arms may be arranged to extend in two planes as desired.

The locking member 58a is shown in more detail in FIG. 10, and comprises a body 88 having a plurality of holes 85 provided therein for securing the locking member 58a to the skull or other region of the body to which the device is to be secured. A channel 87 is provided in the base of the body 88 for receiving the body 80a of the device 80. The channel 87 has a width which is substantially equal to the maximum width of the body 80a, namely the diameter of end 81, such that the body 80a is able to slide within the channel 87.

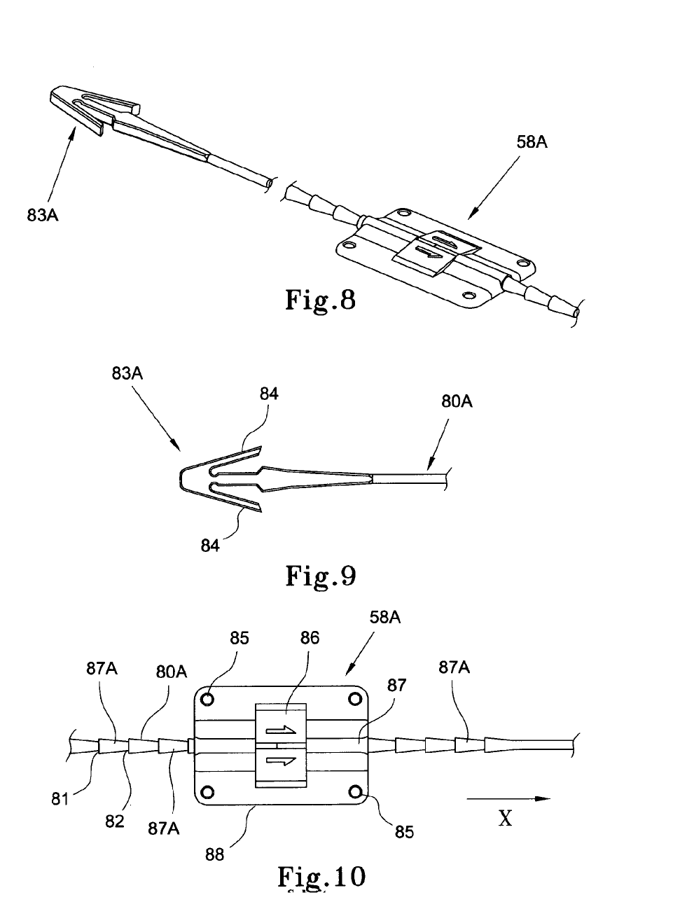

A flexible locking plate 86 is attached to the body 88 to extend orthogonally across the channel 87. In this regard, following positioning of the device 80 and in order to secure the device 80 in final position, the body 80a is inserted in the channel such that it passes under the locking plate 86, in the manner as shown in FIGS. 8 and 10.

Due to the arrangement and configuration of the conical portions 87a along the length of the body 80a, the body is able to slide within the channel 87 in the direction of arrow X, as the flexible locking plate 86 rides over the surface of the conical portions with minimal resistance. As such, when the hook element 83a is embedded within the desired tissue/muscle requiring tightening/lifting, the tissue/muscle can be lifted/tightened by passing and advancing the body 80a through the locking member 58a, thereby causing the tissue/muscle to be pulled in an upward direction. Once the body 80a has been advanced in the direction of arrow X to tighten the device 80, and movement of the body 80a in a direction opposite to arrow X is prevented due to the flexible locking plate 86 coming into contact with the first end 81 of the adjacent conical portion 87a.

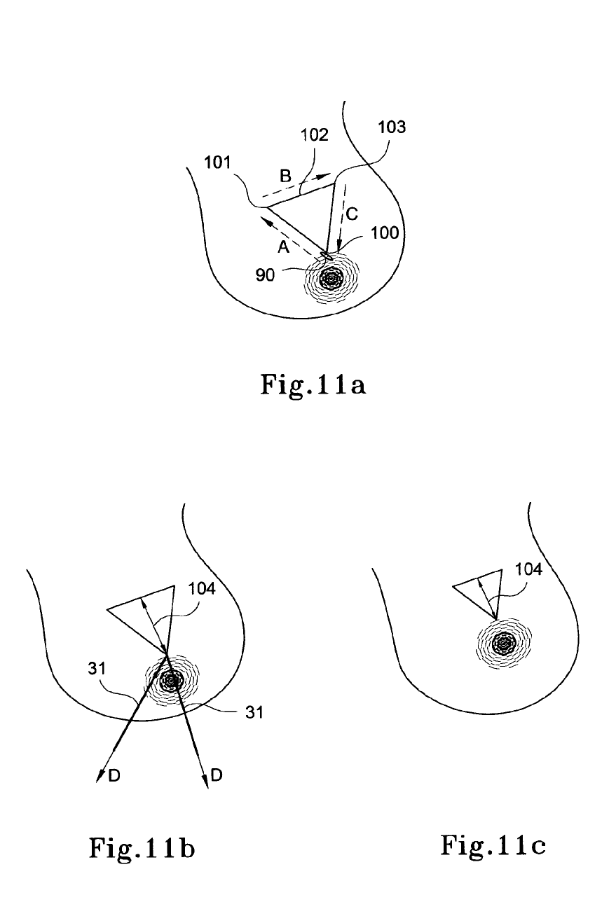

An example of a non surgical mastopexy is illustrated in FIGS. 11a, 11b and 11c.

Prior to surgery, a triangle is drawn on the breast of a patient with the apex 100 of the triangle positioned at the top region of the areola/nipple complex and the base 102 of the triangle at an appropriately spaced distance (depending upon the desired lift). The needle 10 of the invention is inserted through the skin 7 of a patient at entry point 90, at the apex of the triangle 100 and a suture 31 or other implantable device is inserted through the incision and tunnelled in direction A to point 101 of the triangle whereupon the needle is caused to change direction as indicated by arrow B. At point 103, the needle again changes direction and returns to apex 100. The two ends of suture 31 are then pulled in the direction of arrow D. This causes the height 104 of the triangle to reduce as may be seen by a comparison of FIGS. 11b and 11c. The result is that the nipple/areola complex is lifted to the desired degree.

### EXAMPLE

A non surgical mastopexy was performed using the needle of the present invention. A small entry incision was made in the areola and a thread introduced through the incision via the needle. The thread was tunnelled subcutaneously to a region of the breast that was higher than the entry incision. The thread was looped around at said higher region and tunnelled back to the entry incision. Tension was then applied to the thread to lift the nipple/areola complex. A 2-3 cm lift (shift) in the nipple/areola complex was achieved.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A surgical needle comprising:
an elongate body having;
a first end and a second end, each end comprising a skin penetrating tip; said elongate body defining an eye extending at least a majority of the length of the elongate body between said first end and said second end.

2. The surgical needle of claim 1 wherein the length of the eye is greater than or equal to 50% of the length of the elongate body between the two ends.

3. The surgical needle of claim 2 wherein the length of the eye is greater than or equal to 90% of the length of the elongate body between the two ends.

4. The surgical needle of claim 1 wherein the widest diameter of the eye is between 40% and 70% of the total width of the widest part of the elongate body.

5. The surgical needle of claim 1 wherein the widest diameter of the eye is between 20% and 39% of the total width of the widest part of the elongate body.

6. The surgical needle of claim 4 wherein the widest part of the elongate body is mid-way between the two ends.

7. The surgical needle of claim 1 wherein the elongate body comprises depth markers thereon.

8. The surgical needle of claim 7 wherein the depth markers extend from the mid-point of the elongate body and towards both ends.

9. The surgical needle of claim 1 comprising an elongate body made from a material selected from a metal, metal alloy, a polymeric material or a combination thereof.

10. The surgical needle of claim 9 wherein the elongate body is made from surgical grade stainless steel.

11. The surgical needle of claim 1 wherein the elongate body is substantially linear.

12. The surgical needle of claim 1 wherein the elongate body is substantially non-linear.

13. A method of subcutaneously positioning a device within an individual, comprising:
(a) providing a surgical needle comprising an elongate body and a first end and a second end, each end comprising a skin penetrating tip; said elongate body defining an eye extending at least a majority of the length of the elongate body between said first end and said second end;
(b) positioning the device at least partially within the eye of the needle;
(c) subcutaneously introducing the device by passing one of the first end or second end of the needle into and under the skin of the individual;
(d) moving said needle relative to the skin such that said one end exits the skin at a position remote from the position at which the said one end entered and wherein the other of said first and second ends and at least a portion of the device is retained beneath the surface of the skin;
(e) passing said other end of the needle subcutaneously and causing it to exit the skin at a position remote from the position at which the said one end exited the skin of the individual and wherein the said one end and at least a portion of the device is retained beneath the surface of the skin; and
(f) if required, repeating steps (d) and (e) to further subcutaneously advance the device.

14. The method of claim 13 including the step of maneuvering the needle to change the direction of the end that is positioned subcutaneously.

15. A needle assembly comprising:
a surgical needle comprising an elongate body having a first end and a second end, each end comprising a skin penetrating tip; said elongate body defining an eye extending at least a majority of the length of the elongate body between said first end and said second end; and
an implantable device receivable in the eye of the needle.

16. The needle assembly of claim 15 wherein the implantable device comprises a suture.

17. The surgical needle of claim 5 wherein the widest part of the elongate body is mid-way between the two ends.

18. The surgical needle of claim 2 wherein the widest diameter of the eye is between 20% and 39% of the total width of the widest part of the elongate body.

19. The surgical needle of claim 3 wherein the widest diameter of the eye is between 20% and 39% of the total width of the widest part of the elongate body.

20. The surgical needle of claim 2 wherein the widest diameter of the eye is between 40% and 70% of the total width of the widest part of the elongate body.
